(19) United States
(12) Patent Application Publication
(10) Pub. No.: US 2019/0290217 A1
(43) Pub. Date: Sep. 26, 2019

(54) MEDICAL DEVICE ALARM SYSTEMS AND METHODS OF USE

(71) Applicant: Roche Diabetes Care, Inc., Indianapolis, IN (US)

(72) Inventor: James Long, Fishers, IN (US)

(73) Assignee: Roche Diabetes Care, Inc., Indianapolis, IN (US)

(21) Appl. No.: 15/933,642

(22) Filed: Mar. 23, 2018

(51) Int. Cl. A61B 5/00

(52) U.S. Cl. CPC A61B 5/746; G16H 40/60; A61B 5/7465

(57) ABSTRACT

Medical device alarm systems and methods of use including a medical device, a processor, a memory communicatively coupled to the processor, and machine readable instructions stored in the memory may perform at least the following when the instructions are executed by the processor: detect a predefined medical device condition associated with the medical device to determine a detected predefined medical device condition, and generate a high frequency sound alarm based on the detected predefined medical device condition. The high frequency sound alarm may be configured to be tailored to a living entity and be in an upper sound register such that the high frequency sound alarm is configured to alert the living entity capable of hearing the upper sound register of the detected predefined medical device condition.

### TECHNICAL FIELD

The present specification generally relates to medical device alarm systems to generate a sound alarm in response to detection of a medical device condition and, more specifically, to generate a high frequency sound alarm tailored to a living entity in response to detection of the medical device condition and methods of use of such systems.

### BACKGROUND

A medical device alarm system may alert a user to a medical device condition through a visual and/or audible alarms in a normal human hearing range. In instances, these alarms may not be co-located with or enabled on the medical device but may be found in a connected accessory device. In an event that the user is not near the connected accessory device to register and react to the alarm(s) or is otherwise unresponsive to such alarms, a resulting user inaction may lead to serious or even life-threatening medical complications.

Accordingly, a need exists for alternative systems to alert a living entity of a detected medical device condition and methods of use of such systems.

### SUMMARY

In one embodiment, a medical device alarm system may include a medical device, a processor, a memory communicatively coupled to the processor, and machine readable instructions stored in the memory. The machine readable instructions may cause the medical device alarm system to perform at least the following when executed by the processor: detect a predefined medical device condition associated with the medical device to determine a detected predefined medical device condition, and generate a high frequency sound alarm based on the detected predefined medical device condition, wherein the high frequency sound alarm may be configured to be tailored to a living entity and be in an upper sound register such that the high frequency sound alarm is configured to alert the living entity capable of hearing the upper sound register of the detected predefined medical device condition.

In one other embodiment, a medical device alarm system may include a medical device, an accessory device communicatively coupled to the medical device, a processor, a memory communicatively coupled to the processor, and machine readable instructions stored in the memory. The machine readable instructions may cause the medical device alarm system to perform at least the following when executed by the processor: detect a predefined medical device condition associated with the medical device to determine a detected predefined medical device condition, and generate from at least one of the medical device and the accessory device a high frequency sound alarm based on the detected predefined medical device condition. The high frequency sound alarm may be configured to be tailored to a living entity and be in an upper sound register such that the high frequency sound alarm is configured to alert the living entity capable of hearing the upper sound register of the detected predefined medical device condition.

In yet one other embodiment, a method of operating a medical device alarm system may include detecting a predefined medical condition through a signal generated from one of a medical device and an accessory device communicatively coupled to the medical device to define a detected predefined medical condition, and configuring a high frequency sound alarm tailored to a particular living entity. The high frequency sound alarm may be configured to at least one of a predefined high frequency sound range stored in a memory or a user input high frequency sound range, and the particular living entity may be a service animal or a human teenager. The method may further include generating a detected condition alarm for a predefined period of time from at least one of the medical device and the accessory device based on the detected predefined medical condition, generating the high frequency sound alarm in response to failure to silence the detected condition alarm in the predefined period of time through one of a stop selection entry or code entry configured to silence the detected condition alarm, and detecting with a sensor communicatively coupled to the medical device that a corrective therapeutic has been administered to correct the detected predefined medical device condition.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically illustrates a medical device alarm system to generate a high frequency alarm tailored to a hearing range of a living entity, according to one or more embodiments shown and described herein;

FIG. 2 schematically illustrates a system for implementing computer and software based methods to utilize the medical device alarm system of FIG. 1, according to one or more embodiments shown and described herein;

FIG. 3 is a flow chart of a process for using the medical device alarm system of FIG. 1, according to one or more embodiments shown and described herein; and

FIG. 4 is a control scheme setting forth a process for configuring and using the medical device alarm system of FIG. 1, according to one or more embodiments shown and described herein.

### DETAILED DESCRIPTION

Referring generally to the figures, embodiments of the present disclosure are directed to medical device alarm systems to generate a high frequency sound alarm tailored to a living entity in response to detection of the medical device condition and methods of use of such systems. In the medical device alarm systems described herein, generated high frequency sound alarms are configured to alert the living entity of the alarm condition such that the living entity may take action and avoid serious medical complications that could otherwise result from inaction. Such medical device alarm systems improve functionalities of the systems through providing an unconventional technical solution of generation of a particular high frequency sound alarm tailored to a living entity in response to detection of the medical device condition to address a technical problem of diabetes management, for example, while improving the technical field of diabetes management as well as the technology associated with the medical device alarm systems through provision of such novel parameters and algorithms.

The medical device alarm systems described herein may additionally or alternatively generate a high frequency sound alarm to alert a living entity such as a trained service animal or to alert a user that is able to hear the high frequency sound alarm of the alarm condition. The user in at least one embodiment of the present disclosure may be a teenager, or pre-teenager. The user may be, for example, a device user of the medical device or may be capable of assisting the device user to respond to the alarm condition when the device user is unresponsive, such as when the device user is in another room away from a generated alarm, is sleeping, or is otherwise distracted. Thus, medical device(s) and/or optional accessory devices communicatively coupled to the medical device(s) such as connected transceivers, wireless transmitter, mobile phones, or other secondary monitoring devices are equipped with high frequency enabled alarm functions tailored to a living entity to alert the particular living entity of an alarm condition such as a predefined medical device condition as described in greater detail further below.

Reference will now be made in detail to embodiments of the medical device alarm systems, and examples of such systems are illustrated in the accompanying drawings. Wherever possible, the same reference numerals will be used throughout the drawings to refer to the same or like parts. Various embodiments of the medical device alarm systems will be described in further detail herein with specific reference to the appended drawings.

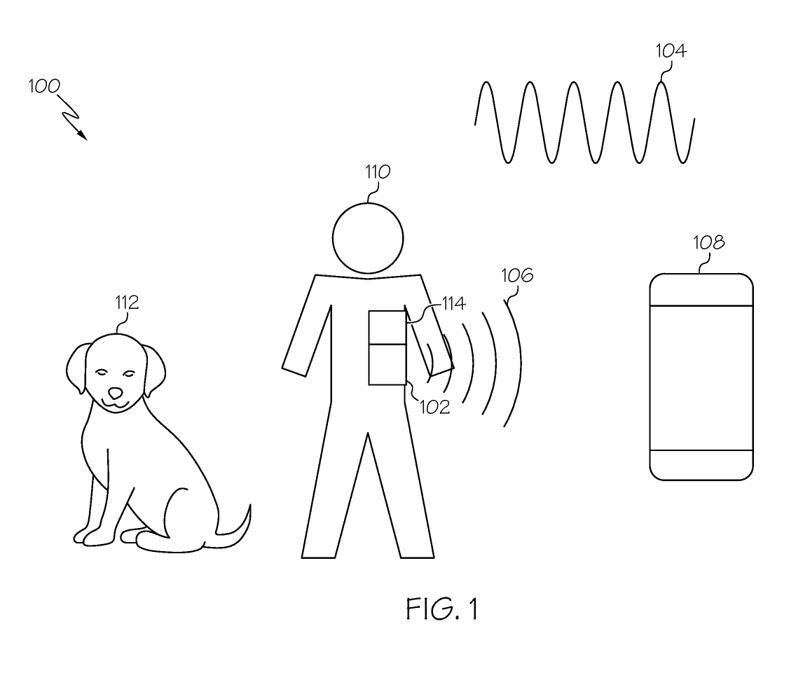

Referring to FIG. 1, a medical device alarm system 100 includes a medical device 102. As an example and not a limitation, the medical device 102 may be a blood glucose (bG) meter, a continuous glucose monitor, another type of medical monitoring device, an insulin delivery device, or other therapeutic delivery device. Further, the medical device 102 may be configured to detect a predefined medical device condition such as, for example, hypoglycemia of a user 110, hyperglycemia of the user 110, a medical device occlusion, and/or a medical device malfunction.

As a non-limiting example, when the medical device 102 is a continuous glucose monitor, the detected predefined medical device condition may include one or more real-time measurements of at least one of hypoglycemia, hyperglycemia, high rates of glucose change above a predefined threshold, one or more calibration warnings, a low battery warning, one or more communication errors, one or more electronic sensor malfunctions, and one or more mechanical malfunctions. When the medical device 102 is associated with an insulin delivery system, the detected predefined medical device condition may include at least one of one or more bolus reminders, one or more infusion set changes, an expiration of a temporary basal rate, one or more insulin cartridge warnings, an occlusion detection, a low battery warning, one or more communication errors, and one or more insulin pump malfunctions. The one or more insulin pump malfunctions may be electronic or mechanical. When the medical device 102 is a bG meter, the detected predefined medical device condition may include at least one of one or more spot monitored measurements of hypoglycemia, one or more spot monitored measurements of hyperglycemia, one or more test measurement reminders, one or more predicted glucose values in a hypoglycemic range, one or more predicted glucose values in a hyperglycemic range, one or more bG calibration alerts for one or more associated continuous glucose monitor devices, a low battery warning, one or more communication errors, and one or more electronic or mechanical bG meter malfunctions.

The medical device 102 may be further configured to generate a high frequency sound alarm 104 based on the detected predefined medical device condition. The high frequency sound alarm 104 is configured to be tailored to a living entity to alert the living entity within a hearing range of the high frequency sound alarm 104 to engage the user 110 of the medical device during an alarm condition to address the alarm condition, particularly if the user is not reacting to an existing alarm mechanism. Further, the high frequency sound alarm 104 is in an upper sound register to alert the living entity capable of hearing the upper sound register of the detected predefined medical device condition. The living entity may be, for example, a service animal such as a dog 112 that is domestically trained or a qualified human such as a teenager. In at least one embodiment, the living entity may be the user 110. As a non-limiting example, the user 110 may be a teenager. A human generally has a hearing range of between 20 Hz to about 20,000 Hz (20 kHz). As a human gets older, the upper level or upper register of the hearing range lowers to around 14-16 kHz. A teenager would tend to have a higher upper range of human that can reach approximately 20 kHz in contrast to a middle-aged person. A dog may have a hearing range that is in a range of from about 40 Hz to 60 kHz.

The qualified human may be a teenager or preteenager in an age range of from about eleven to nineteen years who has an ability to hear the high frequency sound alarm 104. The qualified human may be any human capable of hearing the high frequency sound alarm 104 and responsible enough to respond to the high frequency sound alarm 104. Such hearing capability and/or responsibility capability may be tested and confirmed by the medical device alarm system 100.

As a non-limiting example, the medical device alarm system 100 may include a testing module 114 configured to test and confirm that the qualified human is able to hear the high frequency sound alarm 104. The testing module 114 may further be configured to test and confirm that the qualified human is responsible with respect to responding to the high frequency sound alarm 104 within a predefined period of response time. The testing module 114 may additionally or alternatively be configured to test a qualified animal to confirm that the qualified animal is able to hear the high frequency sound alarm 104 and able to response to the high frequency sound alarm 104 within the predefined period of response time. The testing module 114 may be configured to be applied to test the qualified human and/or qualified animal during a setup period associated with the medical device alarm system 100. Thus, the qualified human and/or qualified animal may be a living entity who has a tested and confirmed hearing ability to hear the high frequency sound alarm 104.

The testing module 114 may be configured to test a range of high frequency sound alarms 104 to confirm that the qualified human and/or qualified animal are capable of hearing and responding to the range of high frequency sound alarms 104 in the predefined period of response time. The medical device alarm system 100 in at least one embodiment is thus able to adjust the high frequency sound alarm 104 in the tested and confirmed range of high frequency sound alarms 104 with respect to the qualified human and/or qualified animal during operation.

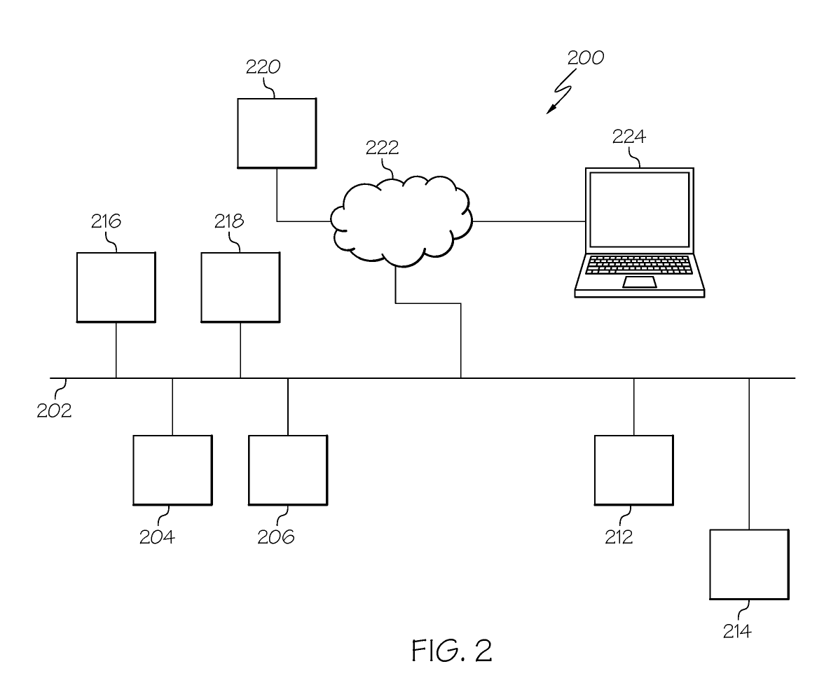

The medical device alarm system 100 may include a processor and a memory communicatively coupled to the processor, such as processor 204 and memory component 206 described in greater detail further below with respect to FIG. 2. The medical device alarm system 100 may further include machine readable instructions stored in the memory that cause the medical device alarm system 100 to perform the instructions when executed by the processor. The instructions may be to detect a predefined medical device condition associated with the medical device 102 to determine a detected predefined medical device condition, and to generate the high frequency sound alarm 104 based on the detected predefined medical device condition.

In various embodiments, the medical device alarm system 100 may be utilized across technology-based platforms and devices. For example, the medical device alarm system 100 may include an accessory device 108 communicatively coupled to the medical device 102. For example, the accessory device 108 may be communicatively coupled to the medical device 102 through a wireless connection 106. In at least one embodiment, the user 110 may use a smart mobile device or other smart device or monitoring device as the accessory device 108 communicatively coupled to the medical device 102 and the medical device alarm system 100. As a smart mobile device such as a smartphone, the accessory device 108 may be enabled with a software application specific to the medical device 102. Thus, when the accessory device 108 is a smart mobile device, the smart mobile device may include a medical device specific application software tool communicatively coupled with the medical device 102. Alternatively, the accessory device 108 may be a wireless transmitter. For example, the medical device alarm system 100 may include instructions to generate the high frequency sound alarm 104 from at least one of the medical device 102 and the accessory device 108. The high frequency sound alarm 104 may be generated by a sound generator component 212, which is described in greater detail below with respect to FIG. 2.

The high frequency sound alarm 104 may be generated from a sound emitter that includes an aerodynamic sound device and/or an electronic sound device. In at least one embodiment, the aerodynamic sound device is a dog whistle, and the electronic sound device is a piezeoelectric emitter. The sound emitter may be configured to produce sound near or above a high human hearing threshold of approximately 20 kHz. Alternatively, the sound emitter may be configured to produce an adjustable high frequency sound alarm 104 in a range of from about 20 kHz to about 100 kHz. Further, the sound emitter may be configured to produce an adjustable high frequency sound alarm 104 based on a type of service animal.

In at least one embodiment, the high frequency sound alarm 104 is associated with one or more frequency selection options configured for selection on a display of the at least one of the medical device 102 and the accessory device 108 that generates the high frequency sound alarm 104. As a non-limiting example, the one or more frequency selection options are configured to present options to tune the high frequency sound alarm 104 to a particular sensitivity of the living entity. The living entity in such a case may be a service animal, and the one or more frequency selection options may be configured to assist in training exercises for the service animal to train the service animal as to one or more meanings associated with the high frequency sound alarm 104.

In at least one embodiment, the high frequency sound alarm 104 may be adjustable through a sound adjustment component 216 as described in greater detail below with respect to FIG. 2. When the living entity is a dog 112, the high frequency sound alarm 104 may be adjustable based on a particular breed of the dog 112. Additionally or alternatively, the high frequency sound alarm 104 may be adjustable based on a particular type of the detected predefined medical device condition. In at least one embodiment, the high frequency sound alarm 104 is adjustable to provide a constant tone or a variable tone. Further, the high frequency sound alarm 104 may be adjustable to provide one of an increasing frequency and a decreasing frequency based on a severity ranking associated with the particular type of the detected predefined medical device condition, such that a higher severity ranking is associated with the increasing frequency and a lower severity ranking lower than the higher severity ranking is associated with the decreasing frequency. The high frequency sound alarm 104 may be adjustable to provide one of an increasing amplitude and a decreasing amplitude based on a severity ranking associated with the particular type of the detected predefined medical device condition, such that a higher severity ranking is associated with the increasing amplitude and a lower severity ranking lower than the higher severity ranking is associated with the decreasing amplitude. The amplitude is associated with a decibel (dB) level of the high frequency sound alarm 104.

The medical device alarm system 100 may include an additional alarm that includes visual and/or audible elements, for example, to notify a user of the detected predefined medical device condition, wherein the audible elements are in a sound register lower than the upper sound register such as in a mid-range hearing register for a human, for example. The additional alarm may be a primary alarm such that, if a user 110 is unresponsive to the primary alarm, the high frequency sound alarm 104 will be generated. Alternatively, the high frequency sound alarm 104 may be generated in addition to the additional alarm. The additional alarm may be generated from the medical device 102 and/or the accessory device 108. The additional alarm and the high frequency sound alarm 104 may be combined into a sound generating unit that includes one or more electronic audio files. The one or more electronic audio files may include one or more sampling rates and tonal frequencies stored in the memory and configured for alarm playback. The audio file(s) may be sampled at a sufficiently high rate to support an associated target playback frequency(s) as defined by the Nyquist sampling theorem. For example, compact disc (CD) audio tracks may include Pulse Code Modulation digital audio files that are sampled at a rate of 44.1 kHz with a depth of 16 bits to support a tonal reproduction of up to 22.05 kHz. Supported higher tonal frequencies would thus require higher sampling rates such as an 88.2 kHz sampling rate to reproduce tones of up to 44.1 kHz. The one or more electronic audio files are configured to produce sound in a frequency range of from about 0 Hz to 100 kHz.

In at least one embodiment, the medical device alarm system 100 includes a detected condition alarm comprising at least one of visual and audible elements in a sound register lower than the upper sound register to notify a user 110 of the detected predefined medical device condition. The high frequency sound alarm 104 may be configured to be triggered after the detected condition alarm is not addressed by a user 110 after a predetermined period of time such that the user 110 is determined to be unresponsive to the detected condition alarm. The high frequency sound alarm 104 may be configured to be set as a primary alarm having priority over the detected condition alarm during a predefined event. The predefined event may be, for example, a time block. The time block may be associated with pre-set sleeping hours and/or may be a time period at which a user 110 of the medical device 102 is at a high risk of alarm non-compliance. As a non-limiting example, predefined event may be a hypoglycemic event during pre-set sleeping hours.

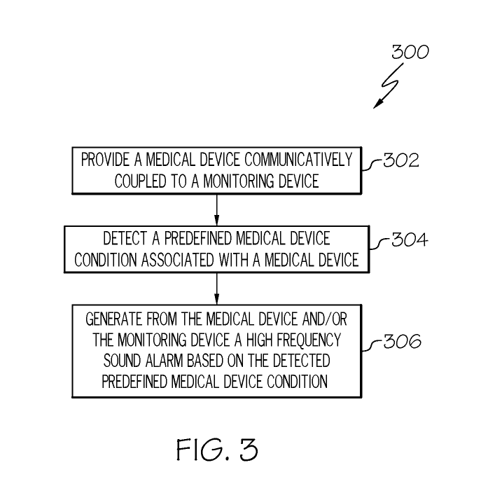

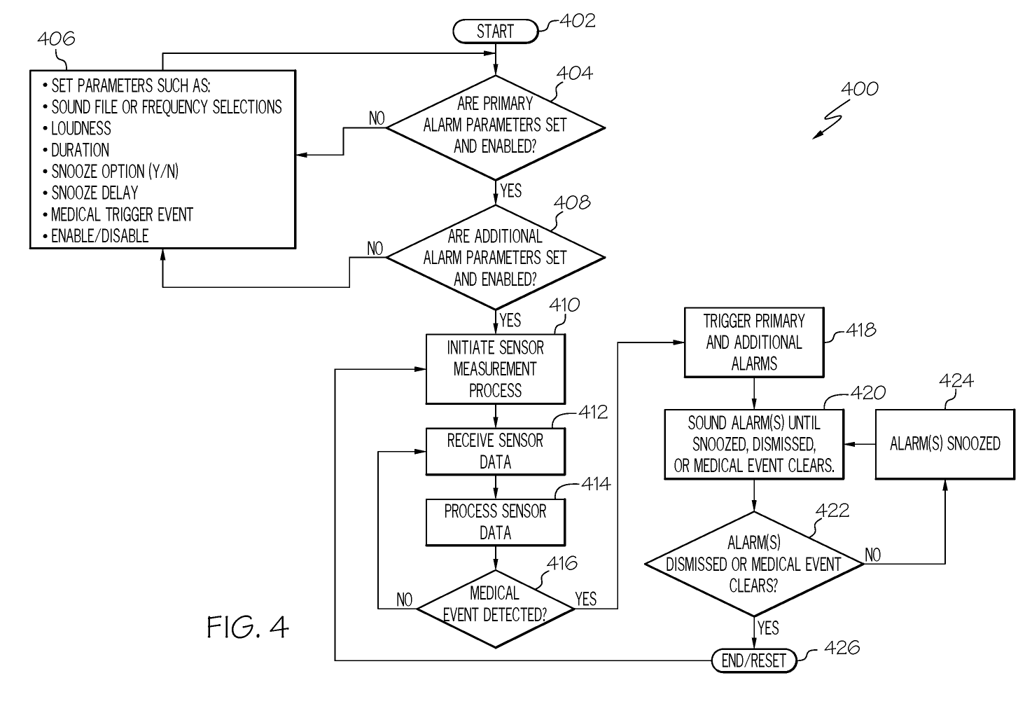

In at least one embodiment, and as described in greater detail below with respect to a process 300 of FIG. 3, a control scheme 400 of FIG. 4 that may be implemented by a processor 204 of FIG. 2, a method of operating a medical device alarm system may include programming logic such as at least one of the process 300 and the control scheme 400 directed to the configuration of the medical device alarm system for a user and/or by a user to generate a high frequency sound alarm 104 tailored to a particular living entity. For example, the logic may involve, as set forth in block 304 of FIG. 3 or block 416 of FIG. 4, detecting a predefined medical condition through a signal generated from one of a medical device 102 and an accessory device 108 communicatively coupled to the medical device 102 to define a detected predefined medical condition as described herein.

The method may further include configuring a high frequency sound alarm 104 tailored to the particular living entity that is a service animal or a human teenager. The high frequency sound alarm 104 may be configured as at least one of a predefined high frequency sound range stored in a memory or as a high frequency sound range input by a user through a graphical user interface (GUI) display screen. As an example and not a limitation, the predefined high frequency sound range may be selected from a list stored in memory associated with a particular type of animal or particular age or hearing ability of the target human such that the particular living entity is capable of hearing the upper range of the predefined high frequency sound range. By way of example and not limitation, the processor 204 may select the predefined high frequency sound range from a list stored in a memory component 206 of FIG. 2 in block 406 of the control scheme 400 of FIG. 4, both of which are described in greater detail further below. Additionally or alternatively, a user may self-input a level or range for the high frequency sound alarm 104 through a GUI display screen on at least one of the medical device 102 and the accessory device 108 or another communicatively coupled device in block 406 of the control scheme 400, for example.

The method may further include generating, as set forth in block 306 of FIG. 3 or block 418 of FIG. 4, a detected condition alarm. The detected condition alarm may be generated through machine-readable instructions executed by the processor 204, for example, for a predefined period of time from at least one of the medical device 102 and the accessory device 108 based on the detected predefined medical condition as described herein. The predefined period of time may be, for example, in a range of from about 2 minutes to about 5 minutes. The high frequency sound alarm 104 may be generated in response to failure to silence the detected condition alarm in the predefined period of time. The silencing may occur in block 420 of FIG. 4, in at least one embodiment, through one of a stop selection entry or a code entry configured to silence the detected condition alarm. For example, a user may silence the detected condition alarm in the predefined period through selecting a stop selection entry on the GUI display screen. Alternatively, the user may silence the detected condition alarm in the predefined period through input of a code entry on the GUI display screen, where the code entry is configured to silence the detected condition alarm. The code entry may be compared to code stored in memory and may silence the alarm based on a match determination of the compared input and stored codes.

In at least one embodiment, a sensor communicatively coupled to the medical device 102 may detect that a corrective therapeutic has been administered to correct the detected predefined medical device condition, such that the detected predefined medical device condition clears as a detected medical event in block 422 of FIG. 4. For example, insulin may be delivered to correct a detected hyperglycemic event, glucagon or sugars may be administered to correct a detected hypoglycemic event, or the sensor may detect that an occlusion within the medical device has been removed. Based on sensor detection of correction of the detected predefined medical device condition, the system may be reset through, for example, manual selection of a system reset option on the GUI display screen or automatically reset through the sensor signal generated based on the sensor detection of correction of the detected predefined medical condition. If the system detects the alarm has been incorrectly silenced such that the detected predefined medical device condition was not addressed, the system may repeat the alarm process after a predefined period of time or present an override option to the user or an option to the user to manually input a confirmation that the detected predefined medical device condition has been addressed.

Referring to FIG. 2, a system 200 for implementing a computer and software-based method to utilize the medical device alarm system 100, as shown in FIG. 1, is illustrated and may be implemented along with using a graphical user interface (GUI) that is accessible at a user workstation (e.g., a computer 224), for example. The system 200 includes a communication path 202, one or more processors 204, a memory component 206, a sound generator component 212, a storage or database 214, an sound adjustment component 216, a network interface hardware 218, a network 222, a server 220, and at least one computer 224. The various components of the system 200 and the interaction thereof will be described in detail below.

While only one application server 220 and one user workstation computer 224 is illustrated, the system 200 can include multiple workstations and application servers containing one or more applications that can be located at geographically diverse locations across a plurality of industrial sites. In some embodiments, the system 200 is implemented using a wide area network (WAN) or network 222, such as an intranet or the Internet, or other wired or wireless communication network that may include a cloud computing-based network configuration (for example, referable to as “the cloud”). The workstation computer 224 may include digital systems and other devices permitting connection to and navigation of the network. Other system 200 variations allowing for communication between various geographically diverse components are possible. The lines depicted in FIG. 2 indicate communication rather than physical connections between the various components.

As noted above, the system 200 includes the communication path 202. The communication path 202 may be formed from any medium that is capable of transmitting a signal such as, for example, conductive wires, conductive traces, optical waveguides, or the like, or from a combination of mediums capable of transmitting signals. The communication path 202 communicatively couples the various components of the system 200. As used herein, the term “communicatively coupled” means that coupled components are capable of exchanging data signals with one another such as, for example, electrical signals via conductive medium, electromagnetic signals via air, optical signals via optical waveguides, and the like.

As noted above, the system 200 includes the processor 204. The processor 204 can be any device capable of executing machine readable instructions. Accordingly, the processor 204 may be a controller, an integrated circuit, a microchip, a computer, or any other computing device. The processor 204 is communicatively coupled to the other components of the system 200 by the communication path 202. Accordingly, the communication path 202 may communicatively couple any number of processors with one another, and allow the modules coupled to the communication path 202 to operate in a distributed computing environment. Specifically, each of the modules can operate as a node that may send and/or receive data.

As noted above, the system 200 includes the memory component 206 which is coupled to the communication path 202 and communicatively coupled to the processor 204. The memory component 206 may be a non-transitory computer readable medium or non-transitory computer readable memory and may be configured as a nonvolatile computer readable medium. The memory component 206 may comprise RAM, ROM, flash memories, hard drives, or any device capable of storing machine readable instructions such that the machine readable instructions can be accessed and executed by the processor 204. The machine readable instructions may comprise logic or algorithm(s) written in any programming language such as, for example, machine language that may be directly executed by the processor, or assembly language, object-oriented programming (OOP), scripting languages, microcode, etc., that may be compiled or assembled into machine readable instructions and stored on the memory component 206. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. In embodiments, the system 200 may include the processor 204 communicatively coupled to the memory component 206 that stores instructions that, when executed by the processor 204, cause the processor to perform one or more functions as described herein.

Still referring to FIG. 2, as noted above, the system 200 comprises the display such as a GUI on a screen of the computer 224 for providing visual output such as, for example, information, graphical reports, messages, or a combination thereof. The computer 224 may include one or more computing devices across platforms, or may be communicatively coupled to devices across platforms, such as mobile smart devices including smartphones, tablets, laptops, and/or the like or medical devices such as blood glucose meters, insulin pumps, continuous glucose monitors, and the like. The display on the screen of the computer 224 is coupled to the communication path 202 and communicatively coupled to the processor 204. Accordingly, the communication path 202 communicatively couples the display to other modules of the system 200. The display can include any medium capable of transmitting an optical output such as, for example, a cathode ray tube, light emitting diodes, a liquid crystal display, a plasma display, or the like. Additionally, it is noted that the display or the computer 224 can include at least one of the processor 204 and the memory component 206. While the system 200 is illustrated as a single, integrated system in FIG. 2, in other embodiments, the systems can be independent systems.

In at least one embodiment, system 200 comprises the sound generator component 212 and a sound adjustment component 216 to generate the high frequency alarm and the sound adjustment component 216 to adjust, for example, the tune, pitch, and/or frequency of the high frequency alarm as described herein. The sound generator component 212 and the sound adjustment component 216 are coupled to the communication path 202 and communicatively coupled to the processor 204. As will be described in further detail below, the processor 204 may process the input signals received from the system modules and/or extract information from such signals.

In at least one embodiment, system 200 includes the network interface hardware 218 for communicatively coupling the system 200 with a computer network such as network 222. The network interface hardware 218 is coupled to the communication path 202 such that the communication path 202 communicatively couples the network interface hardware 218 to other modules of the system 200. The network interface hardware 218 can be any device capable of transmitting and/or receiving data via a wireless network. Accordingly, the network interface hardware 218 can include a communication transceiver for sending and/or receiving data according to any wireless communication standard. For example, the network interface hardware 218 can include a chipset (e.g., antenna, processors, machine readable instructions, etc.) to communicate over wired and/or wireless computer networks such as, for example, wireless fidelity (Wi-Fi), WiMax, Bluetooth, IrDA, Wireless USB, Z-Wave, ZigBee, or the like.

Still referring to FIG. 2, data from various applications running on computer 224 can be provided from the computer 224 to the system 200 via the network interface hardware 218. The computer 224 can be any device having hardware (e.g., chipsets, processors, memory, etc.) for communicatively coupling with the network interface hardware 218 and a network 222. Specifically, the computer 224 can include an input device having an antenna for communicating over one or more of the wireless computer networks described above.

The network 222 can include any wired and/or wireless network such as, for example, wide area networks, metropolitan area networks, the Internet, an Intranet, the cloud, satellite networks, or the like. Accordingly, the network 222 can be utilized as a wireless access point by the computer 224 to access one or more servers (e.g., a server 220). The server 220 and any additional servers generally include processors, memory, and chipset for delivering resources via the network 222. Resources can include providing, for example, processing, storage, software, and information from the server 220 to the system 200 via the network 222. Additionally, it is noted that the server 220 and any additional servers can share resources with one another over the network 222 such as, for example, via the wired portion of the network, the wireless portion of the network, or combinations thereof.

Referring to FIG. 3, in a process 300 for using the medical device alarm system 100 of FIG. 1, a block 302 sets forth that the medical device 102 communicatively coupled to a monitoring device such as the accessory device 108 is provided. In block 304, a predefined medical device condition as described herein and associated with the medical device 102 is detected. In at least one embodiment, the processor 204 is configured to receive input defining the predefined medical device condition through an initial setup phase, for example, as set forth in greater detail below. The processor 204 is then configured to detect the predefined medical device condition in block 304.

In block 306, a high frequency sound alarm 104 based on the detected predefined medical device condition is generated from the medical device 102 and/or the monitoring device such as the accessory device 108 through use of the sound generator component 212 as described herein. The generated sound may be adjusted through use of the sound adjustment component 216 as described herein. In at least one embodiment, the processor 204 is configured to execute instructions to generate the high frequency sound alarm 104 based on the detected predefined medical device condition through the sound generator component 212.

In medical device alarm systems described herein, the high frequency sound alarm 104 generated to alert a living entity such as qualified animal (for example, a trained service animal) or to alert a qualified human (for example, a teenager able to hear the high frequency sound alarm 104) of the alarm condition such as a detected predefined medical device condition as described herein increases the chances for a response to the alarm condition even if a user of the medical device that has the alarm condition is unresponsive to the alarm condition. Increasing the chances of a response to the alarm condition reduces the chance of a serious or even life-threatening medical complication if the alarm condition is not resolved in a suitable amount of time. The ability to tailor the high frequency sound alarm 104 to a living entity may include an ability to adjust the high frequency sound alarm 104 to, for example, a breed of dog or type of service animal in a suitable range for the particular living entity.

A more severe type of alarm condition may trigger a first high frequency sound alarm 104 at a first higher pitch or frequency indicating a higher severity to the living entity in comparison to a less severe type of alarm condition that triggers a second high frequency sound alarm 104 at a second pitch or frequency lower to the first higher pitch or frequency to indicate the lower severity of the alarm condition to the living entity. The living entity may be a service animal trained to understand the different ranges of a plurality of high frequency sound alarms 104 associated with a respective plurality of alarm conditions and to respond to such alarm conditions accordingly. For example, the service animal may respond by waking a sleeping user or by approaching the user in another room to bark or otherwise alert and signal the user of the alarm condition. The high frequency sound alarm 104 may complement a conventional alarm to simultaneously activate and provide an effective alert awareness among human and animal companions to a user of a medical device for effective compliance and response to manage a diabetic condition of the user, or the high frequency sound alarm 104 may be triggered after the conventional alarm is unaddressed by the user after a predetermined period of time such that the user has not attempted to silence or deactivate the conventional alarm.

Referring to FIG. 4, a control scheme 400 setting forth a process for configuring and using the medical device alarm system 100 is illustrated. In at least one embodiment, the processor 204 may be configured to interact with at least one of the medical device 102 and the accessory device 108 to implement the control scheme 400 through executing one or more machine readable instructions directed to the control scheme 400 and stored in the memory component 206. The control scheme 400 starts at in block 402 to proceed with a determination in block 404 of whether primary alarm parameters are set and enabled. If the primary alarm parameters are not set and enabled, the control scheme 400 proceeds to block 406 in which primary alarm parameters are configured and enabled. The primary alarm parameters may be configured and enabled through use of the processor 204 interacting with a GUI, such as the GUI on a screen of the computer 224 of FIG. 2, for example, or a GUI of a mobile device that may be one of the medical device 102, the accessory device 108, or another computing device. Such parameters may include but not be limited to sound file, frequency selections, loudness options, duration options, a snooze option of yes (Y) or no (N), a snooze delay option and time setting, a medical trigger event as the predefined event, predefined medical condition, or predefined medical device condition, and options to enable or disable one or more of the parameters.

After setting and enabling the primary alarm parameters, the control scheme 400 returns to block 404. With a determination in block 404 that the primary alarm parameters are set and enabled, the control scheme 400 proceeds to an optional block 408 to determine whether additional alarm parameters are set and enabled. If the medical device alarm system 100 is configured to include such additional alarm parameters, and if in block 408 the control scheme 400 determines additional alarm parameters are not set and enabled, the control scheme 400 returns to block 406. The additional alarm parameters may additionally be configured and enabled through use of the processor 204 interacting with a GUI, such as the GUI on a screen of the computer 224 of FIG. 2, for example, or a GUI of a mobile device that may be one of the medical device 102, the accessory device 108, or another computing device. Once the additional alarm parameters are configured and enabled in block 406, the control scheme returns and runs through blocks 404, 408, determining that all primary and additional parameters are set and enabled.

The control scheme 400 then advances to block 410 to initialize a sensor measurement process through, for example, a sensor communicatively coupled to the medical device 102 to track and analyze blood glucose and/or other data to determine a medical event as described herein. In at least one embodiment, the processor 204 executes instructions to initialize the sensor measurement process by instructing the sensor to implement an action track and analyze such data, such as instruction a device to take a sample of a user's blood to analyze the user's blood glucose level.

The control scheme 400 receives the sensor data in block 412 and processes the sensor data in block 414. For example, the processor 204 receives an input signal from the sensor regarding the collected data, such as data representative of information regarding the sample of the user's blood, to process the data to determine blood glucose and/or other data based on the input signal. In block 416, the control scheme 400 determines if a medical event is detected. For example, the processor 204 analyzes the input signal from the sensor regarding the collected data to determine and detect the medical event based on the processed data indicative of the determined blood glucose and/or other data and whether the processed data matches rules indicative of an occurrence of the medical event. If not, the control scheme returns to block 412 to loop through blocks 412-416 until the medical event is detected.

Once the medical event is detected in block 416, the control scheme 400 advances to block 418 to trigger the configured and enabled primary and/or additional alarms. In at least one embodiment, the processor 204 receives a signal indicative of an instruction to trigger the selected primary and/or additional alarms associated with a particular medical event based on a signal indicative of detection of the particular medical event. In block 420, the alarm(s) are sounded until the alarm(s) are snoozed, dismissed, or the medical event is cleared. In at least one embodiment, the processor 204 executes instructions to utilize the sound generator component 212 and/or the sound adjustment component 216 to respectively generate and/or adjust the high frequency sound alarm 104 until receiving a signal indicative of the high frequency sound alarm 104 being snoozed by a user, dismissed by a user, or that the medical event has otherwise been cleared and is no longer detected by the processor 204.

In block 422, if a determination is not made that one or more of the alarms are dismissed or the medical event clears, the control scheme 400 advances to block 424 indicating that one or more of the alarms were snoozed and loops through blocks 420-422 until one or more of the alarms are dismissed or the medical event clears. In at least one embodiment, the processor 204 executes instructions to analyze a received signal to determine if the signal is indicative of an alarm dismissal, a clearing of the medical event, or a snoozing event for the one or more alarms. Once one or more of the alarms are dismissed or the medical event clears in block 422 as an appropriate corrective action to the one or more alarms, for example, the control scheme 426 advances to block 426 to end or to reset. If resetting, for example, the control scheme 426 returns to block 410 to loop through blocks 410-426 to detect and respond to subsequent medial events accordingly. In at least one embodiment, the processor 204 executes instructions to implement the appropriate corrective action to clear the medical event, such as through instructions to administer a corrective therapeutic to correct the predefined medical condition as described above. The processor 204 may further execute instructions to reset the control scheme 400 to a desired stage, such as block 410, after receive a sensitive indicative of occurrence of the appropriate corrective action to clear the medical event.

Item 1. A medical device alarm system including a medical device, a processor, a memory communicatively coupled to the processor, and machine readable instructions stored in the memory that cause the medical device alarm system to perform at least the following when executed by the processor: detect a predefined medical device condition associated with the medical device to determine a detected predefined medical device condition, and generate a high frequency sound alarm based on the detected predefined medical device condition, wherein the high frequency sound alarm is configured to be tailored to a living entity and be in an upper sound register such that the high frequency sound alarm is configured to alert the living entity capable of hearing the upper sound register of the detected predefined medical device condition.

Item 2. The medical device alarm system of item 1, wherein the machine readable instructions comprise instructions to generate the high frequency sound alarm from the medical device.

Item 3. The medical device alarm system of items 1 or 2, wherein the medical device alarm system further comprises an accessory device communicatively coupled to the medical device.

Item 4. The medical device alarm system of item 3, wherein the machine readable instruction comprises instructions to generate the high frequency sound alarm from at least one of the medical device and the accessory device.

Item 5. The medical device alarm system of any of items 1 to 4, wherein the living entity is a service animal.

Item 6. The medical device alarm system of any of items 1 to 5, wherein the living entity is a dog.

Item 7. The medical device alarm system of any of items 1 to 6, wherein the high frequency sound alarm is adjustable based on a particular breed of the dog.

Item 8. The medical device alarm system of any of items 1 to 4, wherein the living entity is a teenager.

Item 9. The medical device alarm system of any of items 1 to 8, wherein the medical device is one of a continuous glucose monitor, blood glucose meter, and insulin delivery device.

Item 10. The medical device alarm system of any of items 1 to 9, wherein the detected predefined medical device condition comprises at least one of hypoglycemia, hyperglycemia, a medical device occlusion, and a medical device malfunction.

Item 11. A medical device alarm system including a medical device, an accessory device communicatively coupled to the medical device, a processor, a memory communicatively coupled to the processor, and machine readable instructions stored in the memory that cause the medical device alarm system to perform at least the following when executed by the processor: detect a predefined medical device condition associated with the medical device to determine a detected predefined medical device condition, and generate from at least one of the medical device and the accessory device a high frequency sound alarm based on the detected predefined medical device condition, wherein the high frequency sound alarm is configured to be tailored to a living entity and be in an upper sound register such that the high frequency sound alarm is configured to alert the living entity capable of hearing the upper sound register of the detected predefined medical device condition.

Item 12. The medical device alarm system of item 11, wherein the high frequency sound alarm is generated from a sound emitter comprising at least one of an aerodynamic sound device and an electronic sound device.

Item 13. The medical device alarm system of item 12, wherein the sound emitter is configured to produce an adjustable high frequency sound alarm based on a type of service animal.

Item 14. The medical device alarm system of items 12 or 13, wherein the sound emitter is configured to produce an adjustable high frequency sound alarm in a range of from about 20 kHz to about 100 kHz.

Item 15. The medical device alarm system of any of items 11 to 14, wherein the medical device alarm system is configured to comprise an additional alarm comprising at least one of visual and audible elements to notify a user of the detected predefined medical device condition, wherein the audible elements are in a sound register lower than the upper sound register.

Item 16. The medical device alarm system of item 15, wherein the additional alarm is generated from the accessory device.

Item 17. The medical device alarm system of any of items 11 to 16, wherein the accessory device is a smart mobile device comprising a medical device specific application software tool communicatively coupled with the medical device.

Item 18. The medical device alarm system of any of items 11 to 17, wherein the high frequency sound alarm is adjustable based on a particle type of the detected predefined medical device condition.

Item 19. The medical device alarm system of any of items 11 to 18, wherein the medical device alarm system further comprises a detected condition alarm comprising at least one of visual and audible elements to notify a user of the detected predefined medical device condition, the audible elements are in a sound register lower than the upper sound register, and the high frequency sound alarm is configured to be triggered after the detected condition alarm is not addressed by a user after a predetermined period of time.

Item 20. A method of operating a medical device alarm system, including detecting a predefined medical condition through a signal generated from one of a medical device and an accessory device communicatively coupled to the medical device to define a detected predefined medical condition, configuring a high frequency sound alarm tailored to a particular living entity, the high frequency sound alarm configured to at least one of a predefined high frequency sound range stored in a memory or a user input high frequency sound range, wherein the particular living entity comprises a service animal or a human teenager, generating a detected condition alarm for a predefined period of time from at least one of the medical device and the accessory device based on the detected predefined medical condition, generating the high frequency sound alarm in response to failure to silence the detected condition alarm in the predefined period of time through one of a stop selection entry or code entry configured to silence the detected condition alarm, and detecting with a sensor communicatively coupled to the medical device that a corrective therapeutic has been administered to correct the detected predefined medical device condition.

Item 21. The method of item 20, including the medical device alarm system of any of items 1-19.

Item 22. A processor for a medical device alarm system configured to execute machine readable instructions stored in a memory communicatively coupled to the processor to perform at least the following: detect a predefined medical device condition associated with a medical device to determine a detected predefined medical device condition, and generate a high frequency sound alarm based on the detected predefined medical device condition, wherein the high frequency sound alarm is configured to be tailored to a living entity and be in an upper sound register such that the high frequency sound alarm is configured to alert the living entity capable of hearing the upper sound register of the detected predefined medical device condition.

Item 23. The processor of item 22, wherein the medical device is communicatively coupled to the processor.

Item 24. The processor of item 23, wherein the medical device alarm system is communicatively coupled to the medical device.

Item 25. The processor of any of items 21-24, including the medical device alarm system of any of items 1-19.

Item 26. The method of item 20, including the processor of any of items 21-25.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

It is noted that the terms “substantially” and “about” and “approximately” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A medical device alarm system comprising:
a medical device;
a processor;
a memory communicatively coupled to the processor; and
machine readable instructions stored in the memory that cause the medical device alarm system to perform at least the following when executed by the processor:
detect a predefined medical device condition associated with the medical device to determine a detected predefined medical device condition; and
generate a high frequency sound alarm based on the detected predefined medical device condition, wherein the high frequency sound alarm is configured to be tailored to a living entity and be in an upper sound register such that the high frequency sound alarm is configured to alert the living entity capable of hearing the upper sound register of the detected predefined medical device condition.

2. The medical device alarm system of claim 1, wherein the machine readable instructions comprise instructions to generate the high frequency sound alarm from the medical device.

3. The medical device alarm system of claim 1, wherein the medical device alarm system further comprises an accessory device communicatively coupled to the medical device.

4. The medical device alarm system of claim 3, wherein the machine readable instruction comprises instructions to generate the high frequency sound alarm from at least one of the medical device and the accessory device.

5. The medical device alarm system of claim 1, wherein the living entity is a service animal.

6. The medical device alarm system of claim 1, wherein the living entity is a dog.

7. The medical device alarm system of claim 6, wherein the high frequency sound alarm is adjustable based on a particular breed of the dog.

8. The medical device alarm system of claim 1, wherein the living entity is a teenager.

9. The medical device alarm system of claim 1, wherein the medical device is one of a continuous glucose monitor, blood glucose meter, and insulin delivery device.

10. The medical device alarm system of claim 1, wherein the detected predefined medical device condition comprises at least one of hypoglycemia, hyperglycemia, a medical device occlusion, and a medical device malfunction.

11. A medical device alarm system comprising:
a medical device;
an accessory device communicatively coupled to the medical device;
a processor;
a memory communicatively coupled to the processor; and
machine readable instructions stored in the memory that cause the medical device alarm system to perform at least the following when executed by the processor:
detect a predefined medical device condition associated with the medical device to determine a detected predefined medical device condition; and
generate from at least one of the medical device and the accessory device a high frequency sound alarm based on the detected predefined medical device condition, wherein the high frequency sound alarm is configured to be tailored to a living entity and be in an upper sound register such that the high frequency sound alarm is configured to alert the living entity capable of hearing the upper sound register of the detected predefined medical device condition.

12. The medical device alarm system of claim 11, wherein the high frequency sound alarm is generated from a sound emitter comprising at least one of an aerodynamic sound device and an electronic sound device.

13. The medical device alarm system of claim 12, wherein the sound emitter is configured to produce an adjustable high frequency sound alarm based on a type of service animal.

14. The medical device alarm system of claim 12, wherein the sound emitter is configured to produce an adjustable high frequency sound alarm in a range of from about 20 kHz to about 100 kHz.

15. The medical device alarm system of claim 11, wherein the medical device alarm system is configured to comprise an additional alarm comprising at least one of visual and audible elements to notify a user of the detected predefined medical device condition, wherein the audible elements are in a sound register lower than the upper sound register.

16. The medical device alarm system of claim 15, wherein the additional alarm is generated from the accessory device.

17. The medical device alarm system of claim 16, wherein the accessory device is a smart mobile device comprising a medical device specific application software tool communicatively coupled with the medical device.

18. The medical device alarm system of claim 11, wherein the high frequency sound alarm is adjustable based on a particle type of the detected predefined medical device condition.

19. The medical device alarm system of claim 11, wherein the medical device alarm system further comprises a detected condition alarm comprising at least one of visual and audible elements to notify a user of the detected predefined medical device condition, the audible elements are in a sound register lower than the upper sound register, and the high frequency sound alarm is configured to be triggered after the detected condition alarm is not addressed by a user after a predetermined period of time.

20. A method of operating a medical device alarm system, comprising:
detecting a predefined medical condition through a signal generated from one of a medical device and an accessory device communicatively coupled to the medical device to define a detected predefined medical condition;
configuring a high frequency sound alarm tailored to a particular living entity, the high frequency sound alarm configured to at least one of a predefined high frequency sound range stored in a memory or a user input high frequency sound range, wherein the particular living entity comprises a service animal or a human teenager;
generating a detected condition alarm for a predefined period of time from at least one of the medical device and the accessory device based on the detected predefined medical condition;
generating the high frequency sound alarm in response to failure to silence the detected condition alarm in the predefined period of time through one of a stop selection entry or code entry configured to silence the detected condition alarm; and
detecting with a sensor communicatively coupled to the medical device that a corrective therapeutic has been administered to correct the detected predefined medical device condition.
